(19) United States
(12) Patent Application Publication
(10) Pub. No.: US 2015/0024483 A1
(43) Pub. Date: Jan. 22, 2015

(54) THERAPEUTIC CONDITIONED MEDIA

(71) Applicants: Neil H. Riordan, Trophy Club, TX (US); Jorge Paz Rodriguez, Panama (PA)

(72) Inventors: Neil H. Riordan, Trophy Club, TX (US); Jorge Paz Rodriguez, Panama (PA)

(21) Appl. No.: 14/509,816

(22) Filed: Oct. 8, 2014

Related U.S. Application Data
(62) Division of application No. 13/456,614, filed on Apr. 26, 2012, now abandoned.

(51) Int. Cl. C12N 5/073

(52) U.S. Cl. CPC C12N 5/0605; C12N 2500/90
USPC 435/325

(57) ABSTRACT

Disclosed are therapeutic compositions useful for treatment of degenerative, autoimmune, inflammatory, and neurological conditions. In one embodiment, clinical conditions are treated by administration of a standardized composition of stem cell, progenitor cell, or cellular supernatant. The invention provides doses of conditioned media that mediate therapeutic effects at concentrations that would not be expected to produce biological effects.

### CROSS-REFERENCE TO RELATED APPLICATIONS

This application claims priority to and is a divisional of co-pending U.S. application Ser. No. 13/456,614, filed Apr. 26, 2012, entitled “Therapeutic Conditioned Media”, and U.S. Provisional Application Ser. No. 61/479,359, filed Apr. 26, 2011, entitled “Therapeutic Conditioned Media”, both of which are expressly incorporated herein by reference in their entireties.

### FIELD OF THE INVENTION

The invention pertains to therapeutic compositions. More particularly, the invention relates to compositions with therapeutic properties generated from exposure of a tissue culture media to a cell. More particularly, the invention discloses means and methods resulting in a therapeutic composition useful for the treatment pain, muscle healing/regeneration, articular pain, psoriasis, multiple sclerosis, and rheumatoid arthritis. More specifically, the invention provides specific doses that are capable of inducing biological effects in human conditions.

### BACKGROUND

Stem cells offer significant possibility in the treatment of degenerative diseases. Mesenchymal stem cells (MSC) are a particularly attractive source of stem cells. In addition to “universal donor” properties, MSC have been demonstrated to be capable of differentiating along the orthodox pathways, which includes bone, cartilage, and adipose tissue, as well as along the non-orthodox pathways, including pancreatic, cardiac, neural and hepatic tissues. The original MSC studies isolated cells from the bone marrow. It is believed that the bone marrow MSC function to generate growth factors that support hematopoiesis in the bone marrow microenvironment.

According to the concept that MSC play a physiological function in promoting hematopoiesis, one of the main therapeutical functions of MSC have been to accelerate hematopoietic engraftment. It has been demonstrated that administration of human MSC can accelerate hematopoietic reconstitution in animal models [1, 2]. Accordingly, one of the first clinical uses of MSC has been to accelerate hematopoietic recovery in a 1995 paper by Lazarus et al. who used autologous, in vitro expanded, “mesenchymal progenitor cells” to treat 15 patients suffering from hematological malignancies in remission. The authors demonstrated feasibility of expanding bone marrow derived MSC in vitro. They showed that a 10 milliliter bone marrow sample was capable of 16,000-fold growth over a 4-7 week in vitro culture period. Cell administration was performed in total doses ranging from 1-50×10(6) cells and was not causative of treatment associated adverse effects [3]. In a subsequent study from the same group in 2000, the use of MSC to accelerate hematopoietic reconstitution was performed in a group of 28 breast cancer patients who received high dose chemotherapy. MSC at concentrations of 1-2.2 x 10 (6)/kg were administered intravenously. No treatment associated adverse effects where observed, and leukocytic and thrombocytic reconstitution appeared to undergo “rapid recovery” [4]. It is interesting that these initial uses were actually in patients with neoplasia and no overt acceleration of cancer progression was noted. Besides feasibility, these studies were important because they established the technique for ex vivo expansion and readministration.

Studies along these lines continued which reaffirmed the feasibility of the approach of “repairing stromal” with expanded MSC cells. In 2005, Lazarus et al treated 46 patients suffering from hematological malignancies with HLA-matched allografts comprising bone marrow and donor-derived expanded MSC. The numbers of MSC administered were 1-5 million/kg. On average the time to neutrophil reconstitution as defined by absolute neutrophil count> or =0.500×10(9)/L) and platelet reconstitution as defined by platelet count> or =20×10(9)/L was 14.0 days (range, 11.0-26.0 days) and 20 days (range, 15.0-36.0 days), respectively. Incidence of acute Grade II-IV GVHD was 13/46 and chronic was 22/36 patients that survived for at least 90 days. Relapse of malignancy occurred in 11 patients with a median time to progression of 213.5 days (range, 14-688 days). The authors concluded that cotransplantation of HLA-identical sibling culture-expanded MSCs with an HLA-identical sibling HSC transplant is feasible and seems to be safe, without immediate infusional or late MSC-associated toxicities [5]. These data were of importance since one of the concerns regarding MSC treatment is associated with growth factor production. Given that leukemic patients have minimally residual disease, which seems to be at least in part controlled by recipient immune function [6, 7], the demonstration that recipient did not have an overtly higher incidence of relapse suggests that MSC do not endow a preferential advantage to leukemic cells. This is very interesting given that MSC are generally considered immune suppressive cells [8, 9].

Other studies also supported the safety aspect, and included several variations. For example, Ball et al reported on use of purified donor-specific MSC (1-5 million/kg) being injected alongside with isolated CD34 from HLA-mismatched relatives in 14 pediatric leukemia patients. They showed that in contrast to traditional graft failure rates of 15% in 47 historical controls, all patients given MSCs showed sustained hematopoietic engraftment without any adverse reaction. Interestingly, children given MSCs did not experience more infections compared with controls [10]. Zhang et al [11] reported 12 patients cotransplanted with donor MSC (1.77+/−0.40)×10(6)/kg and HSC. No observable adverse response during and after the infusion of MSCs was reported and hematopoietic reconstitution occurred rapidly. Two patients developed grade II-IV acute GVHD, and two extensive chronic GVHD. Four patients suffered from cytomegalovirus infection but were cured eventually. Up to the time of publication, seven patients have been followed as long as 29-57 months and five patients died. It was concluded by the authors that MSCs can be expanded effectively by culture and it is safe and feasible to cotransplant patients with allogenic culture-expanded MSCs.

Engraftment of cord blood occurs over a more protracted time period as compared to bone marrow. Macmillan et al used parental haploidentical MSC to promote engraftment in 15 pediatric recipients of unrelated donor umbilical cord blood for acute leukemias. Eight patients received MSCs on day 0, with three patients having a second dose infused on day 21. The average dose of the first infusion was 2.1 million/kg (range, 0.9-5.0)/kg, the second infusion was 1 million, 600,000, and 5 million per kg. The reason of the inconsistency was lack of ability to expand cells in vitro. No serious adverse events were observed with any MSC infusion. All eight evaluable patients achieved neutrophil engraftment at a median of 19 days. Probability of platelet engraftment was 75%, at a median of 53 days. At the median follow-up of 6.8 years five patients were alive and disease free [12]. Meuleman et al used donor-derived expanded MSC (10(6)/kg) to treat 6 patients to accelerate hematopoietic recovery. Two patients displayed rapid hematopoietic recovery (days 12 and 21), and four patients showed no response. One patient developed cytomegalovirus (CMV) reactivation 12 days following the MSC infusion and died from CMV disease, although the authors stated that it was impossible to discern whether the reactivation was associated with the MSC therapy or prior immune suppressive regimen [13].

Use of third-party MSC to enhance peripheral blood stem cell grafts was performed by Baron et al in 20 patients who received non-myeloablative hematopoietic stem cell transplant, whose outcomes were compared to a historic control of 16 patients receiving a similar transplant protocol without MSC. MSC were administered half hour to two hours before the hematopoietic graft. Out of the 20 patients, one had primary graft failure. One-year non-relapse mortality was 10%, relapse occurred in 30%, overall survival was 80%, progression-free survival was 60%, and 1-year incidence of death from GVHD or infection with GVHD was 10%. In the historic control group 1-year incidence of non-relapse mortality was 37% (P=0.02), a 1-year incidence of relapse was 25% (NS), a 1-year overall survival and progression free survival was 44% (P=0.02), and 38% (P=0.1), respectively, and a 1-year rate of death from GVHD or infection with GVHD of 31% (P=0.04) [14]. Of particular interest is that the nonmyeloablative protocol used in this study depends largely on donor graft versus leukemia effect [15]. Therefore because the MSC did not cause a greater increase in leukemic relapse, there is suggestion that these cells may not be cancer-promoting, at least not from the perspective of immune suppressive activities. These data suggest that MSC coinfusion may actually possess beneficial properties in terms of graft versus tumor, or at least does not accelerate relapse. However there is some controversy in that Ning et al showed that out of 10 patients who received MSC coinfusion, 6 had relapses, whereas only 3 of the 15 who received transplants without MSC had relapses [16]. There is some debate whether patient selection in the study was appropriately matched between controls and treated groups [17].

Thus the growth factor production features of MSC have used clinically. Another approach has been the use of conditioned media generated by MSC for therapeutic activities. For example, Parekkadan et al. [18] demonstrated that MSC conditioned media has the ability to protect from liver failure. In their study, therapeutic effects on liver failure were observed subsequent to administration of media conditioned by bone marrow MSC. Tissue culture media was concentrated 25 fold and administered intravenously into the penile vein. The therapeutic effects were observed from media conditioned from 2 million cells per rat. Rat weight was approximately 300 grams. Therefore to obtain a therapeutic dose for treatment of a 75 kg human you would need a tissue culture of 500 million cells (250×2 million). Interestingly, this is impossible to generate as a mass therapeutic. Similarly various problems arise from the point that conditioned media would need to be administered intravenously.

Similarly, Theodelina et al [19] used conditioned media from cultures of 500,000 amniotic fluid MSC that was 10 fold concentrated and administered into NOD-SCID mice made ischemic by ligation of the femoral artery. On a per weight basis an average human is 2500× higher in mass than a mouse, therefore the human equivalent would be conditioned media from 1.25 billion cells. In the study, therapeutic effects where observed upon administration of conditioned media 2 times per week for a total of 2 weeks. Such high numbers of cells are extremely difficult to grow for commercial wide-spread human use.

Wei et al [20], used conditioned media from adipose derived cells, concentrated 250× for treatment of a rat model of cerebral palsy. The human concentration equivalent would be approximately 5 billion cells per patient. The use of stem cell conditioned media, however is not without risk. A recent paper [21], demonstrated that conditioned media may have apoptotic effects on neuronal cells in culture, which appeared to be mediated through NMDA and AMPA receptors.

Thus there is a great need for clinically-establishing the feasibility of using conditioned media at a concentration of cells that are relevant to commercialization.

### SUMMARY

The current invention provides methods of generating a therapeutic product based on media conditioned by stem cell populations. In one aspect the invention provides doses of conditioned media that elicit therapeutic effects at concentrations that are capable of eliciting clinical responses, said concentrations being unexpectedly lower than expected.

The following are embodiments herein. A cell population in contact with a liquid media, in which said cell population is in contact with said liquid media for a sufficient time period to endow said liquid media with therapeutic properties for humans or animals. The cell population, further comprising a stressor selected from the group consisting of: a) hypotonic stress; b) hyper or hypothermia; c) culture in media lacking certain nutrients; d) hypoxia and e) culture in media without serum. The cell population, wherein said cell population comprises stem cells selected from the group consisting of a) embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) cord blood stem cells; k) placental stem cells; l) bone marrow stem cells; m) germinal stem cells; n) hair follicle stem cells; o) adipose derived stem cells; p) reprogrammed stem cells; q) peripheral blood derived stem cells; r) peripheral blood mesenchymal stem cells; s) endometrial regenerative cells; t) fallopian tube derived stem cells; u) dermal stem cells; and v) side population stem cells.

The cell population, wherein said placental stem cells are isolated from the placental structure. The cell population, wherein said mesenchymal stem cells are derived from a source selected from the group consisting of: a) bone marrow; b) adipose tissue; c) umbilical cord blood; d) Wharton's Jelly; e) enzymatically digested cord; f) inducible pluripotent generated cells; g) placental tissue; h) peripheral blood mononuclear cells; i) differentiated embryonic stem cells; and j) differentiated progenitor cells. The cell population, wherein said adipose tissue derived stem cells express markers selected from the group consisting of: a) CD13; b) CD29; c) CD44; d) CD63; e) CD73; f) CD90; g) CD166; h) Aldehyde dehydrogenase (ALDH); and i) ABCG2. The cell population, wherein said adipose tissue derived stem cells are a population of purified mononuclear cells extracted from adipose tissue capable of proliferating in culture for more than 1 month. The cell population in contact with a liquid media, wherein said liquid media is selected from the group consisting of: a) alpha MEM; b) DMEM; c) RPMI; d) Opti-MEM; e) IMEM; and f) AIM-V media. The cell population in contact with a liquid media, wherein said cells are expanded in liquid media containing fetal calf serum and subsequently cultured in media substantially lacking said fetal calf serum, with said culture lacking fetal calf serum used for production of a therapeutic product. The cell population in contact with a liquid media, wherein said therapeutic property endowed to said liquid media is ability to inhibit, alleviate, or resolve a condition selected from the group consisting of: a) an inflammatory or autoimmune disorder; b) a disorder associated with, or state of pain; and c) a disorder associated with loss of cells.

The cell population in contact with a liquid media, wherein said inflammatory disorder is selected from the group consisting of: a) multiple sclerosis; b) contact dermatitis; c) psoriasis; d) allergic and non-allergic eye diseases; e) rheumatoid arthritis; f) lupus; g) septic shock; h) radiation overdose; i) copd; j) osteoporosis; k) cognitive disorders; l) Achlorhydra Autoimmune Active Chronic Hepatitis; m) Acute Disseminated Encephalomyelitis; n) Acute hemorrhagic leukoencephalitis; o) Addison's Disease; p) Alopecia areata; q) ALS; r) Fibromyalgia; s) Gastritis; t) Glomerulonephritis; u) Graves' disease; v) Guillain-Barré syndrome; w) Hashimoto's thyroiditis; x) Idiopathic pulmonary fibrosis; y) Scleroderma; z) vitiligo; and aa) diabetes.

The cell population in contact with a liquid media, wherein said disorder associated with a loss of cells is selected from the group consisting of: motor-neurone disease, multiple sclerosis, degenerative diseases of the CNS, dementia, Alzheimer's Disease, Parkinson's Disease, cerebrovascular accidents, epilepsy, temporary ischaemic accidents, mood disorders, psychotic illness, specific lobe dysfunction, pressure related CNS injury, cognitive dysfunction, deafness, blindness, anosmia, motor deficits, sensory deficits, head injury, trauma to the CNS, arrhythmias, myocardial infarction, pericarditis, congestive heart disease, valve related pathologies, myocardial dysfunction, endocardial dysfunction, pericardial dysfunction, sclerosis and thickening of valve flaps, fibrosis of cardiac muscle, decline in cardiac reserve, congenital defects of the heart or circulatory system, developmental defects of the heart or circulatory system, hypoxic or necrotic damage, blood vessel damage, cardiovascular disease (for example, angina, dissected aorta, thrombotic damage, aneurysm, atherosclerosis, emboli damage), disorders of the sweat gland, disorders of the sebaceous gland, piloerectile dysfunction, follicular problems, hair loss, epidermal disease, disease of the dermis or hypodermis, burns, ulcers, sores, infections, striae, seborrhoea, rosacea, port wine stains, disorders of the musculoskeletal system including disease and damage to muscles and bones, endocondral ossification, osteoporosis, osteomalacia, rickets, pagets disease, rheumatism, arthritis, diseases of the endocrine system, diseases of the lymphatic system, diseases of the urinary system, diseases of the reproductive system, metabolic diseases, diseases of the sinus, diseases of the nasopharynx, diseases of the oropharynx, diseases of the laryngopharynx, diseases of the larynx, diseases of the ligaments, diseases of the vocal cords, vestibular folds, glottis, epiglottis, trachea, mucocilliary mucosa, trachealis muscles, emphysema, chronic bronchitis, pulmonary infection, asthma, tuberculosis, cystic fibrosis, diseases of gas exchange, burns, barotraumas, dental care, periodontal disease, deglutination problems, ulcers, enzymatic disturbances/deficiencies, fertility problems, paralysis, dysfunction of absorption or absorptive services, diverticulosis, inflammatory bowel disease, hepatitis, cirrhosis, portal hypertension, diseases of sight, and cancer.

The cell population in contact with a liquid media, wherein said liquid media is concentrated and used for the formulation of a pharmaceutical. The cell population in contact with a liquid media, wherein said formulation generated from said liquid media is administered therapeutically from a group of routes of administration selected from the group consisting of; a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u)bronchially; v) sublingually; w) intranodally; x) by any parenteral route; and y) via inhalation. The cell population in contact with a liquid media, wherein said cell population is immortalized.

The cell population in contact with a liquid media, wherein said cell population is immortalized by means selected from the group consisting of: a) transfection with an oncogene; b) transfection telomerase; and c) transfection with a combination of an oncogene and telomerase. A therapeutic composition useful for treatment of an inflammatory; autoimmune; or degenerative condition, whose activity is mediated, at least in part, through stimulation of cellular regeneration, said composition derived from a liquid media having been in contact with a cell population for a sufficient time point necessary to endow therapeutic activity in said liquid media.

The therapeutic composition, wherein said cell population is selected from a group comprising of a population of cells containing: a) stem cells; b) progenitor cells; and c) differentiated cells. The therapeutic composition, wherein a stressor is added to said cell population. The therapeutic composition, wherein said stressor is selected from the group consisting of: a) hypotonic stress; b) hyper or hypothermia; c) culture in media lacking certain nutrients; d) hypoxia and e) culture in media without serum. The therapeutic composition, wherein said stem cells are selected from the group consisting of: a) embryonic stem cells; b) hematopoietic stem cells; c) mesenchymal stem cells; d) very small embryonic like stem cells; e) inducible pluripotent stem cells; f) bone marrow stem cells; g) amniotic fluid stem cells; h) neuronal stem cells; i) parthenogenically derived stem cells; j) cord blood stem cells; k) placental stem cells; l) bone marrow stem cells; m) germinal stem cells; n) hair follicle stem cells; o) adipose derived stem cells; p) reprogrammed stem cells; q) peripheral blood derived stem cells; r) peripheral blood mesenchymal stem cells; s) endometrial regenerative cells; t) fallopian tube derived stem cells; u) dermal stem cells; and v) side population stem cells.

The therapeutic composition, wherein said therapeutic property endowed to said liquid media is ability to inhibit, alleviate, or resolve a condition selected from the group consisting of: a) an inflammatory or autoimmune disorder; b) a disorder associated with, or state of pain; and c) a disorder associated with loss of cells. The therapeutic composition, wherein said inflammatory disorder is selected from the group consisting of: a) multiple sclerosis; b) contact dermatitis; c) psoriasis; d) allergic and non-allergic eye diseases; e) rheumatoid arthritis; f) lupus; g) septic shock; h) radiation overdose; i) copd; j) osteoporosis; k) cognitive disorders; l) Achlorhydra Autoimmune Active Chronic Hepatitis; m) Acute Disseminated Encephalomyelitis; n) Acute hemorrhagic leukoencephalitis; o) Addison's Disease; p) Alopecia areata; q) ALS; r) Fibromyalgia; s) Gastritis; t) Glomerulonephritis; u) Graves' disease; v) Guillain-Barré syndrome; w) Hashimoto's thyroiditis; x) Idiopathic pulmonary fibrosis; y) Scleroderma; z) vitiligo; and aa) diabetes.

The therapeutic composition, wherein said disorder associated with a loss of cells is selected from the group consisting of: motor-neuron disease, multiple sclerosis, degenerative diseases of the CNS, dementia, Alzheimer's Disease, Parkinson's Disease, cerebrovascular accidents, epilepsy, temporary ischaemic accidents, mood disorders, psychotic illness, specific lobe dysfunction, pressure related CNS injury, cognitive dysfunction, deafness, blindness, anosmia, motor deficits, sensory deficits, head injury, trauma to the CNS, arrhythmias, myocardial infarction, pericarditis, congestive heart disease, valve related pathologies, myocardial dysfunction, endocardial dysfunction, pericardial dysfunction, sclerosis and thickening of valve flaps, fibrosis of cardiac muscle, decline in cardiac reserve, congenital defects of the heart or circulatory system, developmental defects of the heart or circulatory system, hypoxic or necrotic damage, blood vessel damage, cardiovascular disease (for example, angina, dissected aorta, thrombotic damage, aneurysm, atherosclerosis, emboli damage), disorders of the sweat gland, disorders of the sebaceous gland, piloerectile dysfunction, follicular problems, hair loss, epidermal disease, disease of the dermis or hypodermis, burns, ulcers, sores, infections, striae, seborrhoea, rosacea, port wine stains, disorders of the musculoskeletal system including disease and damage to muscles and bones, endocondral ossification, osteoporosis, osteomalacia, rickets, pagets disease, rheumatism, arthritis, diseases of the endocrine system, diseases of the lymphatic system, diseases of the urinary system, diseases of the reproductive system, metabolic diseases, diseases of the sinus, diseases of the nasopharynx, diseases of the oropharynx, diseases of the laryngopharynx, diseases of the larynx, diseases of the ligaments, diseases of the vocal cords, vestibular folds, glottis, epiglottis, trachea, mucocilliary mucosa, trachealis muscles, emphysema, chronic bronchitis, pulmonary infection, asthma, tuberculosis, cystic fibrosis, diseases of gas exchange, burns, barotraumas, dental care, periodontal disease, deglutination problems, ulcers, enzymatic disturbances/deficiencies, fertility problems, paralysis, dysfunction of absorption or absorptive services, diverticulosis, inflammatory bowel disease, hepatitis, cirrhosis, portal hypertension, diseases of sight, and cancer.

The therapeutic composition, wherein said liquid media is concentrated and used for the formulation of a pharmaceutical. The therapeutic composition, wherein said formulation generated from said liquid media is administered therapeutically from a group of routes of administration selected from the group consisting of: a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v)sublingually; w) intranodally; x) by any parenteral route; and y) via inhalation. The therapeutic composition, wherein said cell population is immortalized. The therapeutic composition, wherein said cell population is immortalized by means selected from the group consisting of: a) transfection with an oncogene; b) transfection telomerase; and c) transfection with a combination of an oncogene and telomerase.

A pharmaceutical preparation comprised of a supernatant of a culture that is substantially cell free, said culture comprising of a cell population that is significantly viable in the presence of a tissue culture media, said media being exposed to said cell culture for a period of time sufficient to endow therapeutic properties on said tissue culture media. The pharmaceutical preparation, wherein said media is concentrated in volume. The pharmaceutical preparation, wherein said media is concentrated by means selected from the group consisting of: a) lyophilization and desalting; b) anion exchange chromatography; c) HPLC; d) dialysis; e) use of a filter with a molecular weight cut-off; and f) FPLC. The pharmaceutical preparation, wherein said media is lyophilized and administered sublingually. The pharmaceutical preparation, wherein said media is administered via a route selected from the group consisting of: a) orally; b) intravenously; c)intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v) sublingually; w) intranodally; x) by any parenteral route; and y) via inhalation.

The pharmaceutical preparation, wherein said media is collected from a culture of approximately 30 million Wharton's Jelly mesenchymal cells cultured at approximately 75% confluence in media containing no animal or human products, and no phenol red for a culture period of approximately 24 hours. The pharmaceutical preparation, wherein said media is administered to a patient on an approximate twice per week basis in a volume of 0.5 to 1 ml intramuscularly. The pharmaceutical preparation, wherein said preparation is used for the treatment of pain. The pharmaceutical preparation, wherein said preparation is used for enhancing endurance training, muscle enhancement, and performance enhancement.

The pharmaceutical preparation, wherein said preparation is used for treatment of cachexia. The pharmaceutical preparation, wherein said preparation is admixed either in concentrated or unconcentrated form with an agent suitable for transdermal delivery. The pharmaceutical preparation, wherein said agent suitable for transdermal delivery is an oil selected from the group consisting of: mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C.sub.12-15 alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylangylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semisynthetic derivatives thereof, and combinations thereof.

The pharmaceutical preparation of claim 39, wherein said agent suitable for transdermal delivery is Emu oil.

### DETAILED DESCRIPTION OF THE INVENTION

The invention teaches methods of generating a therapeutic product through growth of various cell populations in a liquid media. In one embodiment, the invention provides a means of creating a medicament useful for the treatment of inflammatory, autoimmune, and degenerative conditions through culturing Wharton Jelly mesenchymal cells in a serum free media. Many types of media may be used and chosen by one of skill in the art. In one embodiment a media is selected from a group comprising of alpha MEM, DMEM, RPMI, Opti-MEM, IMEM, and AIM-V. Cells may be cultured in a variety of media for expansion that contain fetal calf serum, or other growth factors, however, for collection of therapeutic supernatant, in a preferred embodiment, the cells are transferred to a media substantially lacking serum. In some embodiments, the supernatant is administered directly into the patient in need of treatment. It is well known in the art that preparation of the supernatant before administration may be performed by various means, for example, said supernatant may be filter sterilized, or in some conditions concentrated. In a preferred embodiment, the supernatant is administrated intramuscularly in a volume of 0.5 to 1 ml per injection, with two injections per week. In this embodiment a concentration of 30 million Wharton Jelly mesenchymal cells are grown on a plastic surface for approximately 24 hours. Supernatant is harvested, filter sterilized, and stored for administration.

In other embodiments, the conditioned media is used as an active ingredient for the generation of a pharmaceutical formulation. This may comprise administration of the stem cell conditioned media therapeutic agent alone, but preferably comprise administration by way of known pharmaceutical formulations, including tablets, capsules or elixirs for oral administration, suppositories for rectal administration, sterile solutions or suspensions for parenteral or intramuscular administration, liposomal or encapsulated formulations, formulations wherein the therapeutic agent is alone or conjugated to a delivery agent or vehicle, and the like. It will be appreciated that therapeutic entities of the invention will be administered with suitable carriers, excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences (15.sup.th ed, Mack Publishing Company, Easton, Pa. (1975)), particularly Chapter 87 by Blaug, Seymour, therein. These formulations include, for example, powders, pastes, ointments, jellies, waxes, oils, lipids, lipid (cationic or anionic) containing vesicles (such as Lipofectin™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. Any of the foregoing mixtures may be appropriate in treatments and therapies in accordance with the present invention, provided that the active ingredient in the formulation is not inactivated by the formulation and the formulation is physiologically compatible and tolerable with the route of administration. See also Powell et al. “Compendium of excipients for parenteral formulations” PDA J Pharm Sci Technol 52:238-311 (1998) and the citations therein for additional information related to excipients and carriers well known to pharmaceutical chemists. In one embodiment of the invention, one or more agents of the invention are nanoencapsulated into nanoparticles for delivery. The nanoencapsulation material may be biodegradable or nondegradable. The nanoencapsulation materials may be made of synthetic polymers, natural polymers, oligomers, or monomers. Synthetic polymers, oligomers, and monomers include those derived from polyalkyleneoxide precursor molecules, such as poly(ethylene oxide) (PEO), poly(ethylene glycol) (PEG) and copolymers with poly(propylene oxide) (PEG-co-PPO), poly (vinyl alcohol) (PVA), poly(vinylpyrrolidone) (PVP), poly(ethyloxazoline) (PEOX), polyaminoacids, and pseudopolyamino acids, and copolymers of these polymers. Sawhney et al., Macromolecules 26:581-587 (1993). Copolymers may also be formed with other water-soluble polymers or water insoluble polymers, provided that the conjugate is water soluble. An example of a water-soluble conjugate is a block copolymer of polyethylene glycol and polypropylene oxide, commercially available as a Pluronic™ surfactant (BASF). Natural polymers, oligomers and monomers include proteins, such as fibrinogen, fibrin, gelatin, collagen, elastin, zein, and albumin, whether produced from natural or recombinant sources, and polysaccharides, such as agarose, alginate, hyaluronic acid, chondroitin sulfate, dextran, dextran sulfate, heparin, heparin sulfate, heparan sulfate, chitosan, gellan gum, xanthan gum, guar gum, water soluble cellulose derivatives, and carrageen. These polymers are merely exemplary of the types of nanoencapsulation materials that can be utilized and are not intended to represent all the nanoencapsulation materials within which entrapment is possible. In one embodiment, the therapeutic agent is administered in a topical formulation. Topical formulations are useful in the treatment of conditions associated with dermal diseases. For example, topical administration of stem cell conditioned media may be performed for the treatment of psoriasis, scleroderma, or acne. Topical forms of administration may consist of, for example, aqueous and nonaqueous gels, creams, multiple emulsions, microemulsions, liposomes, ointments, aqueous and nonaqueous solutions, lotions, aerosols, skin patches, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer. Topical formulations of the invention may include a dermatologically acceptable carrier, e.g., a substance that is capable of delivering the other components of the formulation to the skin with acceptable application or absorption of those components by the skin. The carrier will typically include a solvent to dissolve or disperse the therapeutic agent, and, optionally one or more excipients or other vehicle ingredients. Carriers useful in accordance with the topical formulations of the present invention may include, by way of non-limiting example, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). Topical formulations of the invention are prepared by mixing a compound of the invention with a topical carrier according to well-known methods in the art, for example, methods provided by standard reference texts e.g., Remington: The Science and Practice of Pharmacy, 1577-1591, 1672-1673, 866-885 (Alfonso R. Gennaro ed. 19th ed. 1995); and Ghosh et al., Transdermal and Topical Drug Delivery Systems (1997). In other embodiments, moisturizers or humectants, sunscreens, fragrances, dyes, and/or thickening agents such as paraffin, jojoba, PABA, and waxes, surfactants, occlusives, hygroscopic agents, emulsifiers, emollients, lipid-free cleansers, antioxidants and lipophilic agents, may be added to the topical formulations of the invention if desired. A topical formulation of the invention may be designed to be left on the skin and not washed shortly after application. Alternatively, the topical formulation may be designed to be rinsed off within a given amount of time after application.

In one embodiment, the treatment of immunological diseases is performed by administration of the stem cell conditioned media directly to its site of therapeutic activity, which in the case of many immune diseases is in the lymph nodes. For example, the therapeutic agent may be injected directly into the lymph nodes. Preferred lymph nodes for intranodal injections of inhibitors of T cell-dependent activation are the major lymph nodes located in the regions of the groin, the underarm and the neck. In another embodiment, the therapeutic agent is administered distal to the site of its therapeutic activity.

In one aspect of the invention, potency of the conditioned media product may be quantified by use of assessing protein production. Such assays are well-known to one of skill in the art. Following the teachings of Jiao et al. [22], production of IL-10 may be quantified. For quantification of anti-inflammatory activity, the term “inflammation” will be understood by those skilled in the art to include any condition characterized by a localized or a systemic protective response, which may be elicited by physical trauma, infection, chronic diseases, such as those mentioned above, and/or chemical and/or physiological reactions to external stimuli (e.g., as part of an allergic response). Any such response, which may serve to destroy, dilute or sequester both the injurious agent and the injured tissue, may be manifested by, for example, heat, swelling, pain, redness, dilation of blood vessels and/or increased blood flow, invasion of the affected area by white blood cells, loss of function and/or any other symptoms known to be associated with inflammatory conditions. The term “inflammation” will thus also be understood to include any inflammatory disease, disorder or condition per se, any condition that has an inflammatory component associated with it, and/or any condition characterized by inflammation as a symptom, including, inter alia, acute, chronic, ulcerative, specific, allergic and necrotic inflammation, and other forms of inflammation known to those skilled in the art. The term thus also includes, for the purposes of this invention, inflammatory pain and/or fever caused by inflammation.

In another embodiment, conditioned media is generated in an ex-vivo extracorporal setting. Specifically, cells of interest are grown on the outside of a hollow-fiber filter which is connected to a continuous extracorporeal system. Said hollow-fiber system contains pores in the hollow fiber of sufficient size so has to allow exchange of proteins between circulating blood cells and cultured cells on the outside of the hollow fibers, without interchange of host cells with said stem cells.

In one embodiment, stem cell conditioned media is used in combination with an immune suppressive agent to augment its activity. While stem cell conditioned media may be used alone for treatment and/or maintenance of disease remission, in some embodiments coadministration with an immune suppressive agent may be required. Additionally, an immune suppressive agent may be useful for “induction therapy”. Depending on disease and response desired, it will be known to one of skill in the art to choose from various immune suppressive agents. For example, some immune suppressive agents, such as anti-CD52 antibodies may be used when a systemic depletion of T and B cells is desired, whereas agents that concurrently stimulate T regulatory cell activity, such as Rapamycin, may be desired in other cases. The skilled practitioner is guided to several agents that are known in the art for causing immune suppression, which include cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-.alpha. inhibitors, TNF-.alpha. sequestration agents, and methotrexate. More specifically, anti-inflammatory agents may comprise one or more of, e.g., anti-TNF-.alpha., lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidoc aproic acid, s-adenosylmethionine, 3-amino-4-hydroxybutyric acid, amixetrine, bendazac, benzydamine, .alpha.-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid, disodium 3-succinyloxy-beta-glycyrrhetinate, etc.

When selecting stem cells for use in the practice of the current invention, several factors must be taken into consideration, such as: ability for ex vivo expansion without loss of ability to secrete therapeutic factors, ease of extraction, and general potency of activity. Ex vivo expansion ability of stem cells can be measured using typical proliferation and colony assays known to one skilled in the art, while identification of therapeutic activity depends on functional assays that test biological activities such as: ability to support endothelial function, ability to protect neurons from degeneration/atrophy, and induce proliferation of endogenous stem cells. Assessment of therapeutic activity can also be performed using surrogate assays which detect markers associated with a specific therapeutic activity. Such markers include CD34 or CD133, which are associated with stem cell activity and ability to support angiogenesis [23]. Other assays useful for identifying therapeutic activity of stem cell populations for use with the current invention include evaluation of production of factors associated with the therapeutic activity desired. For example, identification and quantification of production of FGF, VEGF, angiopoietin, or other such angiogenic molecules may be used to serve as a guide for approximating and quantifying growth factor/anti-apoptotic factors elaborated by said cells into culture media [24].

For quantification of effects that stem cells have on conditioned media, and therefore a quantification of the potency of conditioned media, one needs to first decide the therapeutic indication sought. If one seeks to utilize conditioned media for immune suppression, one may assess levels of immune modulatory components in said conditioned media. Examples of soluble immune suppressive factors include: IL-4 [25], IL-10 [26], IL-13 [27], TGF-b [28], soluble TNF-receptor [29], and IL-1 receptor agonist [30]. Membrane-bound immunoinhibitor molecules that may be shed by stem cells and therefore another marker for quantification of specific therapeutic properties: HLA-G [31], FasL [32], PD-1L [33], Decay Accelerating Factor [34], and membrane-associated TGF-b [35]. Enzymes whose biological activity causes alteration in supernatant composition to possess immune suppressive activities include indolamine 2,3 dioxygenase [36] and arginase type II [37]. In order to optimize desired immune suppressive ability, a wide variety of assays are known in the art, including mixed lymphocyte culture, ability to generate T regulatory cells in vitro, and ability to inhibit natural killer or CD8 cell cytotoxicity. In situations where increased angiogenic potential of said conditioned media therapeutic product is desired, assessment of proteins associated with stimulation of angiogenesis may be performed. These include VEGF[38], FGF1 [39], FGF2 [40], FGF4 [41], FrzA [42], and angiopoietin [43]. In some situations the cells in contact with media that generate conditioned media may be transfected with genes to allow for enhanced cellular viability, anti-apoptotic genes suitable for transfection may include bc1-2 [44], bcl-xl [45], and members of the XIAP family [46]. Alternatively it may be desired to increase the proliferative lifespan of said mesenchymal stem cells through transfection with enzymes associated with anti-senescence activity. Said enzymes may include telomerase or histone deacetylases.

In one embodiment mesenchymal cells are generated through culture and subsequently culture media is used for generation of a therapeutic composition. Said therapeutic composition is preferably generated in a medium that is free from human or animal products, with said medium also lacking phenol red. For extraction and growth of mesenchymal stem cells, the skilled practitioner of the invention is referred to examples known in the literature, which include U.S. Pat. No. 5,486,359 describing methods for culturing such and expanding mesenchymal stem cells, as well as providing antibodies for use in detection and isolation. Additionally, U.S. Pat. No. 5,942,225 teaches culture techniques and additives for differentiation of such stem cells which can be used in the context of the present invention to produce increased numbers of cells with ability to secrete agents that possess angiogenic activities. Although U.S. Pat. No. 6,387,369 teaches use of mesenchymal stem cells for regeneration of cardiac tissue, we believe that in accordance with published literature [47, 48] stem cells generated through these means are actually angiogenically potent and therefore may be utilized in the context of the current invention. Without being bound to a specific theory or mechanism of action, it appears that mesenchymal stem cells induce angiogenesis through production of factors such as vascular endothelial growth factor, hepatocyte growth factor, adrenomedullin, and insulin-like growth factor-1 [49], quantification of said growth factors may be useful in standardizing doses in the preparation of said stem cell conditioned media therapeutic product.

Historically, MSC are obtained from bone marrow sources for clinical use, although this source may have disadvantages because of the invasiveness of the donation procedure and the reported decline in number of bone marrow derived mesenchymal stem cells during aging. Alternative sources of mesenchymal stem cells include adipose tissue [50], placenta and Wharton's Jelly [51, 52], scalp tissue [53] and cord blood [54]. While mesenchymal stem cells generated from bone marrow, cord blood, and adipose tissue appear to possess similar morphology and phenotype, ability to induce colony formation appears to be highest using stem cells from adipose tissue and interestingly in contrast to bone marrow and adipose derived mesenchymal cells, only the cord blood derived cells lacked ability to undergo adipocyte differentiation. Within the context of the current invention, our data suggests that conditioned media generated using Wharton's Jelly as a source of cells possesses unique characteristics in contrast to adipose-derived stem cells. It is also known that the proliferative potential appears to be the highest with cord blood mesenchymal stem cells which were capable of expansion to approximately 20 times, whereas cord blood cells expanded an average of 8 times and bone marrow derived cells expanded 5 times [55]. Accordingly, one skilled in the art will understand that mesenchymal stem cells for use with the present invention may be selected upon individual patient characteristics and the end result sought.

For use in the context of the present invention, embryonic stem cells possess certain desirable properties, which include unique “early” growth factor production profile. It is believed in the art that many of the therapeutic effects of ES cell administration are mediated by paracrine factors. This is promising since therapeutic use of ES cells themselves is limited by formation of teratomas [56]. Another embodiment of the current invention is the use of embryonic stem cell supernatant as a therapeutic product. Specific embodiments include identification of substantially purified fractions of said supernatant capable of inducing endothelial cell proliferation, smooth muscle regeneration, and/or neuronal cell proliferation/survival, and/or anti-inflammatory activity, and/or stimulation of endogenous reparative processes. Identification of such therapeutically active fractions may be performed using methods commonly known to one skilled in the art, and includes separation by molecular weight, charge, affinity towards substrates and other physico-chemical properties. In one particular embodiment, supernatant of embryonic stem cell cultures is harvested substantially free from cellular contamination by use of centrifugation or filtration. Supernatant may be concentrated using means known in the art such as solid phase extraction using C18 cartridges (Mini-Spe-ed C18-14%, S.P.E. Limited, Concord ON). Said cartridges are prepared by washing with methanol followed by deionized-distilled water. Up to 100 ml of embryonic stem cell supernatant may be passed through each cartridge before elution. After washing the cartridges material adsorbed is eluted with 3 ml methanol, evaporated under a stream of nitrogen, redissolved in a small volume of methanol, and stored at 4.degree. C. Before testing the eluate for activity in vitro, the methanol is evaporated under nitrogen and replaced by culture medium. Said C18 cartridges are used to adsorb small hydrophobic molecules from the embryonic stem cell culture supernatant, and allows for the elimination of salts and other polar contaminants. It may, however be desired to use other adsorption means in order to purify certain compounds from the embryonic stem cell supernatant. Said concentrated supernatant may be assessed directly for biological activities useful for the practice of this invention, or may be further purified. Further purification may be performed using, for example, gel filtration using a Bio-Gel P-2 column with a nominal exclusion limit of 1800 Da (Bio-Rad, Richmond Calif.). Said column may be washed and pre-swelled in 20 mM Tris-HCl buffer, pH 7.2 (Sigma) and degassed by gentle swirling under vacuum. Bio-Gel P-2 material be packed into a 1.5.times.54 cm glass column and equilibrated with 3 column volumes of the same buffer. Embryonic stem cell supernatant concentrates extracted by C18 cartridge may be dissolved in 0.5 ml of 20 mM Tris buffer, pH 7.2 and run through the column. Fractions may be collected from the column and analyzed for biological activity. Other purification, fractionation, and identification means are known to one skilled in the art and include anionic exchange chromatography, gas chromatography, high performance liquid chromatography, nuclear magnetic resonance, and mass spectrometry. Administration of supernatant active fractions may be performed locally or systemically. For the practice of the invention, the practitioner is referred to the numerous methods of generating embryonic stem cells that are known in the art. Patents describing the generation of embryonic stem cells include U.S. Pat. No. 6,506,574 to Rambhatla, U.S. Pat. No. 6,200,806 to Thomson, U.S. Pat. No. 6,432,711 to Dinsmore, and U.S. Pat. No. 5,670,372 to Hogan.

In one embodiment of the invention, embryonic stem cells are differentiated into endothelial progenitor cells in vitro, followed by administration of conditioned media from these cells to a patient in need of therapy at a concentration and frequency sufficient to induce a therapeutic response. Differentiation into endothelial progenitors may be performed by several means known in the art [57]. One such means includes generation of embryoid bodies through growing human embryonic stem cells in a suspension culture. Said embryoid bodies are subsequently dissociated and cells expressing endothelial progenitor markers are purified [58]. Purification of endothelial cells from embryoid bodies can be performed using, of example, selection for PECAM-1 expressing cells. Another alternative method of generating endothelial progenitors for use in generation of conditioned media from embryonic stem cells involves removing media from embryonic stem cells a period of time after said embryonic stem cells are plated and replacing said media with a media containing endothelial-differentiating factors. For example, after plating of embryonic stem cells for a period between 6 and 48 hours, but more preferably between 20 and 24 hours, the original media in which embryonic stem cells were cultured is washed off the cells and endothelial cell basal medium-2 (EBM2), with 5% fetal calf serum, VEGF, bFGF, IGF-1, EGF, and ascorbic acid is added to the cells. This combination is commercially available (EGM2-MV Bullet Kit; Clonetics/BioWhittaker, Walkersville, Md.). By culturing the embryonic stem cells for 20-30 days in the EGM2 medium, with changing of media every 3 to 5 days, a population of endothelial progenitors can be obtained. For such cells to be useful in the practice of the present invention, functionality of growth factors produced by said endothelial precursors, and their differentiated progeny must be assessed. Methods of assessing stimulation of angiogenesis are well known in the art [59].

For the practice of the invention supernatants generated by culture with cells may be administered to the patient in an injection solution, which may be saline, mixtures of autologous plasma together with saline, or various concentrations of albumin with saline. Ideally pH of the injection solution is from about 6.4 to about 8.3, optimally 7.4. Excipients may be used to bring the solution to isotonicity such as, 4.5% mannitol or 0.9% sodium chloride, pH buffers with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes. Injection can be performed systemically, or more specifically, via routes of administration selected from; a) orally; b) intravenously; c) intramuscularly; d) intraperitoneally; e) intrathecally; f) alimentarily; g) intraspinally; h) intra-articularly; i) intra-joint; j) subcutaneously; k) buccally; l) vaginally; m) rectally; n) dermally; o) transdermally; p) ophthalmically; q) auricularly; r) mucosally; s) nasally; t) tracheally; u) bronchially; v) sublingually; w) intranodally; x) by any parenteral route; and y) via inhalation.

In one particular method, cord blood cells are used for generation of conditioned media, said cord blood is collected from fresh placenta and mononuclear cells are purified by centrifugation using a density gradient such as Ficoll or Percoll, in another method cord blood mononuclear cells are isolated from contaminating erythrocytes and granulocytes by the Hetastarch with a 6% (wt/vol) hydroxyethyl starch gradient. Cells are subsequently washed to remove contaminating debris, assessed for viability, and admixed with culture media to generate a conditioned media. As described within this application, conditioned media is ideally generated for practice within the current invention by a 24 hour culture, however one of skill in the art may identify other time points without deviated from the spirit of the invention. In another embodiment of the invention, cord blood stem cells are fractionated and the fraction with enhanced therapeutic activity is administered to the patient. Enrichment of cells with therapeutic activity may be performed using physical differences, electrical potential differences, differences in uptake or excretion of certain compounds, as well as differences in expression marker proteins. Distinct physical property differences between stem cells with high proliferative potential and low proliferative potential are known. Accordingly, in some embodiments of the invention, it will be useful to select cord blood stem cells with a higher proliferative ability, whereas in other situations, a lower proliferative ability may be desired. In embodiments of the invention where specific cellular physical properties are the basis of differentiating between cord blood stem cells with various biological activities, discrimination on the basis of physical properties can be performed using a Fluorescent Activated Cell Sorter (FACS), through manipulation of the forward scatter and side scatter settings. Other methods of separating cells based on physical properties include the use of filters with specific size ranges, as well as density gradients and pheresis techniques. When differentiation is desired based on electrical properties of cells, techniques such as electrophotoluminescence may be used in combination with a cell sorting means such as FACS. Selection of cells based on ability to uptake certain compounds can be performed using, for example, the ALDESORT system, which provides a fluorescent-based means of purifying cells with high aldehyde dehydrogenase activity. Cells with high levels of this enzyme are known to possess higher proliferative and self-renewal activities in comparison to cells possessing lower levels. Other methods of identifying cells with high proliferative activity includes identifying cells with ability to selectively efflux certain dyes such as rhodamine-123 and or Hoechst 33342. Without being bound to theory, cells possessing this property often express the multidrug resistance transport protein ABCG2, and are known for enhanced regenerative ability compared to cells which do not possess this efflux mechanism. In other embodiments cord blood cells are purified for certain therapeutic properties based on expression of markers. In one particular embodiment, cord blood cells are purified for the phenotype of endothelial precursor cells. Said precursors, or progenitor cells express markers such as CD133, and/or CD34. Said progenitors may be purified by positive or negative selection using techniques such as magnetic activated cell sorting (MACS), affinity columns, FACS, panning, or by other means known in the art. Cord blood derived endothelial progenitor cells may be administered directly into the target tissue for ED, or may be administered systemically. Another variation of this embodiment is the use of differentiation of said endothelial precursor cells in vitro, followed by infusion into a patient. Verification for endothelial differentiation may be performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

Certain desired activities can be endowed onto said cord blood stem cells prior to using as a source of cells for generation of conditioned media. In one specific embodiment cord blood cells may be “activated” ex vivo by a brief culture in hypoxic conditions in order to upregulate nuclear translocation of the HIF-1 transcription factor and endow said cord blood cells with enhanced production of angiogenic growth factors. Hypoxia may be achieved by culture of cells in conditions of 0.1% oxygen to 10% oxygen, preferably between 0.5% oxygen and 5% oxygen, and more preferably around 1% oxygen. Cells may be cultured for a variety of timepoints ranging from 1 hour to 72 hours, more preferably from 13 hours to 59 hours and more preferably around 48 hours. Assessment of angiogenic, and other desired activities useful for the practice of the current invention, can be performed during optimization of conditioned media production. In addition to induction of hypoxia, other therapeutic properties can be endowed unto cord blood stem cells through treatment ex vivo with factors such as dedifferentiating compounds, proliferation inducing compounds, or compounds known to endow and/or enhance cord blood cells to possess properties useful for the practice of the current invention. In one embodiment cord blood cells are cultured with an inhibitor of the enzyme GSK-3 in order to enhance expansion of cells with pluripotent characteristics while not increasing the rate of differentiation. In another embodiment, cord blood cells are cultured in the presence of a DNA methyltransferase inhibitor such as 5-azacytidine in order to endow a “de-differentiation” effect. In another embodiment cord blood cells are cultured in the presence of a differentiation agent that skews said cord blood stem cells to generate enhance numbers of cells which are useful for generation of conditioned media.

In contrast to cord blood stem cells, placental stem cells may be purified directly from placental tissues, said tissues including the chorion, amnion, and villous stroma [51, 60]. In another embodiment of the invention, placental tissue is mechanically degraded in a sterile manner and treated with enzymes to allow dissociation of the cells from the extracellular matrix. Such enzymes include, but not restricted to trypsin, chymotrypsin, collagenases, elastase and/or hylauronidase. Suspension of placental cells are subsequently washed, assessed for viability, and may either be used directly for the practice of the invention. Alternatively, cells may be purified for certain populations with increased biological activity. Purification may be performed using means known in the art, and described above for purification of cord blood stem cells, or may be achieved by positive selection for the following markers: SSEA3, SSEA4, TRA1-60, TRA1-81, c-kit, and Thy-1. In some situations it will be desirable to expand cells before use for generation of conditioned media. Expansion can be performed by culture ex vivo with specific growth factors [61, 62]. The various embodiments of the invention described above for cord blood and embryonic stem cells can also be applied for placental stem cells.

Bone marrow stem cells may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest” [63]. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. Additionally, ex vivo expansion and/or selection may also be utilized for augmentation of desired biological properties for use in creation of conditioned media. The various embodiments of the invention described above for cord blood and embryonic stem cells can also be applied for bone marrow stem cells.

Amniotic fluid is routinely collected during amniocentesis procedures. One method of practicing the current invention is utilizing amniotic fluid derived stem cells for generation of conditioned media. In one embodiment amniotic fluid mononuclear cells are utilized therapeutically in an unpurified manner or heterogeneous manner. Said amniotic fluid stem cells are used to endow therapeutic properties on media. In other embodiments amniotic fluid stem cells are substantially purified based on expression of markers such as SSEA-3, SSEA4, Tra-1-60, Tra-1-81 and Tra-2-54, and subsequently administered. In other embodiments cells are cultured, as described in US patent application # 20050054093, expanded, and subsequently used for production of conditioned media. Amniotic stem cells are described in the following references [64-66]. One particular aspect of amniotic stem cells that makes them amenable for use in practicing certain aspects of the current invention is their bi-phenotypic profile as being both mesenchymal and neural progenitors [67]. This property is useful for treatment of patients with conditions associated with neurological dysfunction.

Stem cells committed to the neuronal lineage, or neuronal progenitor cells, are used in the practice of some specific embodiments of the invention. Said cells may be generated by differentiation of embryonic stem cells, may be freshly isolated from fetal tissue (ie mesencephalic), may be generated by transdifferentiation, or by reprogramming of a cell. Neuronal progenitors are selected by use of markers such as polysialyated N-CAM, N-CAM, A2B5, nestin and vimentin. The various embodiments of the invention described above for cord blood and embryonic stem cells can also be applied for neuronal stem cells.

A wide variety of stem cells are known to circulate in the periphery. These include multipotent, pluripotent, and committed stem cells. In some embodiments of the invention mobilization of stem cells is induced in order to increase the number of circulating stem cells, so that harvesting efficiency is increased. Said mobilization allows for harvest of cells with desired properties for practice of the invention without the need to perform bone marrow puncture. A variety of methods to induce mobilization are known. Methods such as administration of cytotoxic chemotherapy, for example, cyclophosphamide or 5-fluoruracil are effective but not preferred in the context of the current invention due to relatively unacceptable adverse events profile. Suitable agents useful for mobilization include: granulocyte colony stimulating factor (G-CSF), granulocyte-macrophage colony stimulating factor (GM-CSF), interleukin 1 (IL-1), interleukin 3 (IL-3), stem cell factor (SCF, also known as steel factor or kit ligand), vascular endothelial growth factor (VEGF), Flt-3 ligand, platelet-derived growth factor (PDGF), epidermal growth factor (EGF), fibroblast growth factor-1 (FGF-1), fibroblast growth factor-2 (FGF-2), thrombopoietin (TPO), interleukin-11 (IL-11), insulin-like growth factor-1 (IGF-1), megakaryocyte growth and development factor (MGDF), nerve growth factor (NGF), hyperbaric oxygen, and 3-hydroxy-3-methyl glutaryl coenzyme A (HMG CoA)reductase inhibitors. The various embodiments of the invention described above for cord blood and embryonic stem cells can also be applied for circulating peripheral blood stem cells.

Adipose derived stem cells express markers such as CD9; CD29 (integrin beta 1); CD44 (hyaluronate receptor); CD49d,e (integrin alpha 4, 5); CD55 (decay accelerating factor); CD105 (endoglin); CD106 (VCAM-1); CD166 (ALCAM). These markers are useful not only for identification but may be used as a means of positive selection, before and/or after culture in order to increase purity of the desired cell population. In terms of purification and isolation, devices are known to those skilled in the art for rapid extraction and purification of cells adipose tissues. U.S. Pat. No. 6,316,247 describes a device which purifies mononuclear adipose derived stem cells in an enclosed environment without the need for setting up a GMP/GTP cell processing laboratory so that patients may be treated in a wide variety of settings. One embodiment of the invention involves attaining 10-200 ml of raw lipoaspirate, washing said lipoaspirate in phosphate buffered saline, digesting said lipoaspirate with 0.075% collagenase type I for 30-60 min at 37° C. with gentle agitation, neutralizing said collagenase with DMEM or other medium containing autologous serum, preferably at a concentration of 10% v/v, centrifuging the treated lipoaspirate at approximately 700-2000 g for 5-15 minutes, followed by resuspension of said cells in an appropriate medium such as DMEM. Cells are subsequently washed and cultured for 24 hours in DMEM media.

### EXAMPLE 1

### Production of Conditioned Media

Human umbilical cords were obtained from healthy mothers in our hospital after they gave their informed consent. Umbilical cords were processed within 4 h and stored at 4° C. in sterile saline until use. The cords were rinsed several times in sterile phosphate-buffered saline (PBS) to remove blood components and cut into small pieces (2-3 cm). Cord vessels (2 arteries and 1 vein) were removed to avoid endothelial cell contamination. The Wharton's jelly parts of the cord were cut into pieces 0.5-1 cm3 and placed directly into 75-cm2 flasks for culture expansion in low-glucose Dulbecco's modified Eagle's medium (LG-DMEM) containing 10% fetal bovine serum (FBS), 100 U/ml penicillin/streptomycin at 37° C., and 5% (v/v) CO2. Cells were detached with 0.05% trypsin-EDTA and reseeded in new culture flasks. When cells reached 75% confluence, they were washed with PBS and serum free, phenol-free DMEM media was added for 24 hours to a total of approximately 30 million cells. Supernatant was collected, filter sterilized with a 0.2 micron filter and either frozen or lyophilized for further use.

### EXAMPLE 2

### Performance Enhancement

24 athletes are administered 1 ml of conditioned media prepared as described in Example 1 intramuscularly 2 times per week for a period of 2 months, whereas 24 athletes of similar body mass and physical shape are administered placebo. Subsequent to strength and cardiovascular training over the 2 month period, the 24 athletes receiving conditioned media demonstrated a statistically significant increase in post-exercise recovery time and improvement of both cardiovascular and weight-lifting ability as compared to the placebo control patients.

A 52 year old male received sublingual administration of conditioned media, the lyophilized equivalent of 1 ml of conditioned media, 2 times per week for a period of 1 month. The subject underwent a marked reduction in existing pains and was capable of swimming 1 kilometer, whereas before initiating intake of conditioned media was only capable of swimming 100 meters. The subject reported increased energy and clarity of mind. Additionally, the subject reported resolution of prior muscle and joint pains, especially resolution of knee pain within 2 weeks of receiving conditioned media.

### EXAMPLE 3

### Reduction of Osteoarthritis

Administration of liquid conditioned media directly into muscles adjacent to the metatarsal on a 2 times per week basis of 1 ml of conditioned media generated as described in Example 1 resulted in resolution of arthritic pain. Additionally administration of a similar dose of conditioned media resulted in reduction in pain subsequent to an ACL injury. Administration into another patient of conditioned media into joints via the intraarticular route, of 1 ml conditioned media, resulted in patient reduction.

### EXAMPLE 4

### Reduction of Disc Degeneration Associated Pain/Back Pain

Two patients, a male and female, received conditioned media prepared as described in Example 1, in muscles adjacent to lumbar area of pain origination. Administration was performed two times per week. Pain resolution was observed within 3 days after initial administration. Both patients reported a marked reduction, or complete giving up, of pain medications subsequent to receiving stem cell conditioned media. Another patient who was 62 years old was treated with conditioned media as described above and very little, if any, back pain secondary to back surgery received was perceived.

### EXAMPLE 5

### Reduction in Psoriasis

Topical administration of conditioned media admixed with Emu oil on a patient with psoriasis resulted in amelioration of pain and resolution of psoriatic lesions. Concentration of conditioned media was 1 ml per dose, two doses a week.

### EXAMPLE 6

### Reduction in Autoimmunity

Two patients with multiple sclerosis who were entering relapse were treated by twice weekly administration of 0.5-1 ml of conditioned media administered subcutaneously. Therapeutic effects were observed within the first week of treatment, including regaining balance, clarity, and overall feeling of improved health. One patient was in a whellchair and underwent such a profound recover that they started walking with a walker. Another patient with uveritis was administered conditioned media according to the above mentioned protocol. The patient underwent recovery. A patient with scleroderma was treated for 2 months using stem cell conditioned media, a highly potent resolution of disease was observed.

### EXAMPLE 7

### Reduction in Polymyalgia Rheumatica

A patient suffering from polymyalgia rheumatica was administered stem cell conditioned media intramuscularly twice per week at a volume of 0.5-1 ml generated as described in Example 1. Prior to treatment the patient reported systemic pain, weakness and fatigue. The patient also developed a severe diverticulitis and required a hemicolectomy. Subsequent to receiving conditioned media a rapid healing of surgical lesions was observed along with a reduction in pain scores. Importantly, the feelings of pain, weakness and fatigue resolved.

### REFERENCES

1. Almeida-Porada, G., et al., Cotransplantation of human stromal cell progenitors into preimmune fetal sheep results in early appearance of human donor cells in circulation and boosts cell levels in bone marrow at later time points after transplantation. Blood, 2000. 95(11): p. 3620-7.

2. Noort, W. A., et al., Mesenchymal stem cells promote engraftment of human umbilical cord blood-derived CD34(+) cells in NOD/SCID mice. Experimental hematology, 2002. 30(8): p. 870-8.

3. Lazarus, H. M., et al., Ex vivo expansion and subsequent infusion of human bone marrow-derived stromal progenitor cells (mesenchymal progenitor cells): implications for therapeutic use. Bone marrow transplantation, 1995. 16(4): p. 557-64.

4. Koc, O. N., et al., Rapid hematopoietic recovery after coinfusion of autologous-blood stem cells and culture-expanded marrow mesenchymal stem cells in advanced breast cancer patients receiving high-dose chemotherapy. Journal of clinical oncology: official journal of the American Society of Clinical Oncology, 2000. 18(2): p. 307-16.

5. Lazarus, H. M., et al., Cotransplantation of HLA-identical sibling culture-expanded mesenchymal stem cells and hematopoietic stem cells in hematologic malignancy patients. Biology of blood and marrow transplantation: journal of the American Society for Blood and Marrow Transplantation, 2005. 11(5): p. 389-98.

6. Oka, T., et al., Evidence for specific immune response against P210 BCR-ABL in long-term remission CML patients treated with interferon. Leukemia: official journal of the Leukemia Society of America, Leukemia Research Fund, U.K, 1998. 12(2): p. 155-63.

7. Pulsipher, M. A., et al., Allogeneic transplantation for pediatric acute lymphoblastic leukemia: the emerging role of peritransplantation minimal residual disease/chimerism monitoring and novel chemotherapeutic, molecular, and immune approaches aimed at preventing relapse. Biology of blood and marrow transplantation: journal of the American Society for Blood and Marrow Transplantation, 2009. 15(1 Suppl): p. 62-71.

8. Griffin, M. D., T. Ritter, and B. P. Mahon, Immunological aspects of allogeneic mesenchymal stem cell therapies. Human gene therapy, 2010. 21(12): p. 1641-55.

9. Xue, Q., et al., The negative co-signaling molecule b7-h4 is expressed by human bone marrow-derived mesenchymal stem cells and mediates its T-cell modulatory activity. Stem cells and development, 2010. 19(1): p. 27-38.

10. Ball, L. M., et al., Cotransplantation of ex vivo expanded mesenchymal stem cells accelerates lymphocyte recovery and may reduce the risk of graft failure in haploidentical hematopoietic stem-cell transplantation. Blood, 2007. 110(7): p. 2764-7.

11. Zhang, X., et al., Cotransplantation of HLA-identical mesenchymal stem cells and hematopoietic stem cells in Chinese patients with hematologic diseases. International journal of laboratory hematology, 2010. 32(2): p. 256-64.

12. Macmillan, M. L., et al., Transplantation of ex-vivo culture-expanded parental haploidentical mesenchymal stem cells to promote engraftment in pediatric recipients of unrelated donor umbilical cord blood: results of a phase I-II clinical trial. Bone marrow transplantation, 2009. 43(6): p. 447-54.

13. Meuleman, N., et al., Infusion of mesenchymal stromal cells can aid hematopoietic recovery following allogeneic hematopoietic stem cell myeloablative transplant: a pilot study. Stem cells and development, 2009. 18(9): p. 1247-52.

14. Baron, F., et al., Cotransplantation of mesenchymal stem cells might prevent death from graft-versus-host disease (GVHD) without abrogating graft-versus-tumor effects after HLA-mismatched allogeneic transplantation following nonmyeloablative conditioning. Biology of blood and marrow transplantation: journal of the American Society for Blood and Marrow Transplantation, 2010. 16(6): p. 838-47.

15. McSweeney, P. A., et al., Hematopoietic cell transplantation in older patients with hematologic malignancies: replacing high-dose cytotoxic therapy with graft-versus-tumor effects. Blood, 2001. 97(11): p. 3390-400.

16. Ning, H., et al., The correlation between cotransplantation of mesenchymal stem cells and higher recurrence rate in hematologic malignancy patients: outcome of a pilot clinical study. Leukemia: official journal of the Leukemia Society of America, Leukemia Research Fund, U.K, 2008. 22(3): p. 593-9.

17. Behre, G., et al., Reply to ‘The correlation between cotransplantation of mesenchymal stem cells and higher recurrence rates in hematologic malignancy patients: outcome of a pilot clinical study’ by Ning et al. Leukemia: official journal of the Leukemia Society of America, Leukemia Research Fund, U.K, 2009. 23(1): p. 178; author reply 179-80.

18. Parekkadan, B., et al., Mesenchymal stem cell-derived molecules reverse fulminant hepatic failure. PloS one, 2007. 2(9): p. e941.

19. Teodelinda, M., et al., Amniotic liquid derived stem cells as reservoir of secreted angiogenic factors capable of stimulating neo-arteriogenesis in an ischemic model. Biomaterials, 2011. 32(15): p. 3689-99.

20. Wei, X., et al., IFATS collection: The conditioned media of adipose stromal cells protect against hypoxia-ischemia-induced brain damage in neonatal rats. Stem cells, 2009. 27(2): p. 478-88.

21. Horn, A. P., et al., Conditioned medium from mesenchymal stem cells induces cell death in organotypic cultures of rat hippocampus and aggravates lesion in a model of oxygen and glucose deprivation. Neuroscience research, 2009. 63(1): p. 35-41.

22. Jiao, J., et al., A mesenchymal stem cell potency assay. Methods in molecular biology, 2011. 677: p. 221-31.

23. Higashi, Y., et al., Oxidative stress, endothelial function and angiogenesis induced by cell therapy and gene therapy. Curr Pharm Biotechnol, 2006. 7(2): p. 109-16.

24. Tonnesen, M. G., X. Feng, and R. A. Clark, Angiogenesis in wound healing. J Investig Dermatol Symp Proc, 2000. 5(1): p. 40-6.

25. Jansen, J. H., et al., Interleukin-4. A regulatory protein. Blut, 1990. 60(5): p. 269-74.

26. Zhou, X., et al., Boosting interleukin-10 production: therapeutic effects and mechanisms. Curr Drug Targets Immune Endocr Metabol Disord, 2005. 5(4): p. 465-75.

27. Mentink-Kane, M. M. and T. A. Wynn, Opposing roles for IL-13 and IL-13 receptor alpha 2 in health and disease. Immunol Rev, 2004. 202: p. 191-202.

28. Kriegel, M. A., et al., Transforming growth factor-beta: recent advances on its role in immune tolerance. Curr Rheumatol Rep, 2006. 8(2): p. 138-44.

29. Fernandez-Botran, R., F. A. Crespo, and X. Sun, Soluble cytokine receptors in biological therapy. Expert Opin Biol Ther, 2002. 2(6): p. 585-605.

30. Dayer, J. M., Evidence for the biological modulation of IL-1 activity: the role of IL-1Ra. Clin Exp Rheumatol, 2002. 20(5 Suppl 27): p. S14-20.

31. Rouas-Freiss, N., et al., HLA-G proteins in cancer: do they provide tumor cells with an escape mechanism? Cancer Res, 2005. 65(22): p. 10139-44.

32. Bohana-Kashtan, O. and C. I. Civin, Fas ligand as a tool for immunosuppression and generation of immune tolerance. Stem Cells, 2004. 22(6): p. 908-24.

33. Okazaki, T. and T. Honjo, The PD-1-PD-L pathway in immunological tolerance. Trends Immunol, 2006. 27(4): p. 195-201.

34. Longhi, M. P., et al., Holding T cells in check—a new role for complement regulators? Trends Immunol, 2006. 27(2): p. 102-8.

35. Wahl, S. M., J. M. Orenstein, and W. Chen, TGF-beta influences the life and death decisions of T lymphocytes. Cytokine Growth Factor Rev, 2000. 11(1-2): p. 71-9.

36. Mellor, A. L. and D. H. Munn, IDO expression by dendritic cells: tolerance and tryptophan catabolism. Nat Rev Immunol, 2004. 4(10): p. 762-74.

37. Serafini, P., I. Borrello, and V. Bronte, Myeloid suppressor cells in cancer: recruitment, phenotype, properties, and mechanisms of immune suppression. Semin Cancer Biol, 2006. 16(1): p. 53-65.

38. Matsumoto, R., et al., Vascular endothelial growth factor-expressing mesenchymal stem cell transplantation for the treatment of acute myocardial infarction. Arterioscler Thromb Vasc Biol, 2005. 25(6): p. 1168-73.

39. Klein, S., M. Roghani, and D. B. Rifkin, Fibroblast growth factors as angiogenesis factors: new insights into their mechanism of action. Exs, 1997. 79: p. 159-92.

40. Chen, C. H., et al., Fibroblast growth factor 2: from laboratory evidence to clinical application. Curr Vasc Pharmacol, 2004. 2(1): p. 33-43.

41. Grines, C., et al., Angiogenic gene therapy with adenovirus 5 fibroblast growth factor-4 (Ad5FGF-4): a new option for the treatment of coronary artery disease. Am J Cardiol, 2003. 92(9B): p. 24N-31N.

42. Dufourcq, P., et al., FrzA, a secreted frizzled related protein, induced angiogenic response. Circulation, 2002. 106(24): p. 3097-103.

43. Morisada, T., et al., Angiopoietins and angiopoietin-like proteins in angiogenesis. Endothelium, 2006. 13(2): p. 71-9.

44. Murphy, E., K. Imahashi, and C. Steenbergen, Bcl-2 regulation of mitochondrial energetics. Trends Cardiovasc Med, 2005. 15(8): p. 283-90.

45. Harada, H. and S. Grant, Apoptosis regulators. Rev Clin Exp Hematol, 2003. 7(2): p. 117-38.

46. Guegan, C., et al., PTD-XIAP protects against cerebral ischemia by anti-apoptotic and transcriptional regulatory mechanisms. Neurobiol Dis, 2006. 22(1): p. 177-86.

47. Caplan, A. I. and J. E. Dennis, Mesenchymal stem cells as trophic mediators. J Cell Biochem, 2006.

48. Shyu, K. G., et al., Mesenchymal stem cells are superior to angiogenic growth factor genes for improving myocardial performance in the mouse model of acute myocardial infarction. J Biomed Sci, 2006. 13(1): p. 47-58.

49. Nagaya, N., et al., Transplantation of mesenchymal stem cells improves cardiac function in a rat model of dilated cardiomyopathy. Circulation, 2005. 112(8): p. 1128-35.

50. Knippenberg, M., et al., Adipose tissue-derived mesenchymal stem cells acquire bone cell-like responsiveness to fluid shear stress on osteogenic stimulation. Tissue Eng, 2005. 11(11-12): p. 1780-8.

51. Portmann-Lanz, C. B., et al., Placental mesenchymal stem cells as potential autologous graft for pre- and perinatal neuroregeneration. Am J Obstet Gynecol, 2006. 194(3): p. 664-73.

52. Zhang, X., et al., Mesenchymal progenitor cells derived from chorionic villi of human placenta for cartilage tissue engineering. Biochem Biophys Res Commun, 2006. 340(3): p. 944-52.

53. Shih, D. T., et al., Isolation and characterization of neurogenic mesenchymal stem cells in human scalp tissue. Stem Cells, 2005. 23(7): p. 1012-20.

54. Kadivar, M., et al., In vitro cardiomyogenic potential of human umbilical vein-derived mesenchymal stem cells. Biochem Biophys Res Commun, 2006. 340(2): p. 639-47.

55. Kern, S., et al., Comparative Analysis of Mesenchymal Stem Cells from Bone Marrow, Umbilical Cord Blood or Adipose Tissue. Stem Cells, 2006.

56. Vogel, G., Cell biology. Ready or not? Human ES cells head toward the clinic. Science, 2005. 308(5728): p. 1534-8.

57. Levenberg, S., Engineering blood vessels from stem cells: recent advances and applications. Curr Opin Biotechnol, 2005. 16(5): p. 516-23.

58. Trounson, A., The production and directed differentiation of human embryonic stem cells. Endocr Rev, 2006. 27(2): p. 208-19.

59. Gerecht-Nir, S., et al., Human embryonic stem cells as an in vitro model for human vascular development and the induction of vascular differentiation. Lab Invest, 2003. 83(12): p. 1811-20.

60. Demir, R., et al., Classification of human placental stem villi: review of structural and functional aspects. Microsc Res Tech, 1997. 38(1-2): p. 29-41.

61. Mohamed, A. A., et al., Ex vivo expansion of stem cells: defining optimum conditions using various cytokines. Lab Hematol, 2006. 12(2): p. 86-93.

62. Kashiwakura, I. and T. A. Takahashi, Fibroblast growth factor and ex vivo expansion of hematopoietic progenitor cells. Leuk Lymphoma, 2005. 46(3): p. 329-33.

63. Mineishi, S., [Immunobiology of mini-transplant]. Nippon Rinsho, 2003. 61(9): p. 1489-94.

64. Bossolasco, P., et al., Molecular and phenotypic characterization of human amniotic fluid cells and their differentiation potential. Cell Res, 2006. 16(4): p. 329-36.

65. Sartore, S., et al., Amniotic mesenchymal cells autotransplanted in a porcine model of cardiac ischemia do not differentiate to cardiogenic phenotypes. Eur J Cardiothorac Surg, 2005. 28(5): p. 677-84.

66. Prusa, A. R., et al., Neurogenic cells in human amniotic fluid. Am J Obstet Gynecol, 2004. 191(1): p. 309-14.

67. Tsai, M. S., et al., Clonal amniotic fluid-derived stem cells express characteristics of both mesenchymal and neural stem cells. Biol Reprod, 2006. 74(3): p. 545-51.

All references cited herein are expressly incorporated by reference in their entireties.

1. An adherent mesenchymal cell population isolated from Wharton's Jelly, cultured in contact with a liquid media lacking fetal calf serum, in which contact of said liquid media with said cells endows said liquid media with ability to reduce pain subsequent to administration.

2. The adherent mesenchymal cell population of claim 1 wherein said liquid media is phenol free and serum free DMEM.

3. The adherent mesenchymal cell population of claim 2, wherein said cells are in contact with said liquid media for a period of time ranging from 2 hrs to 72 hours.

4. The adherent mesenchymal cell population of claim 1, wherein said administration is performed sublingually.

5. An adherent mesenchymal cell population isolated from Wharton's Jelly, cultured in contact with a liquid media lacking fetal calf serum, in which contact of said liquid media with said cells endows said liquid media with ability to enhance muscle mass in a subject after said subject performs athletic exercises subsequent to administration.

6. The adherent mesenchymal cell population of claim 5 wherein said liquid media is phenol free and serum free DMEM.

7. The adherent mesenchymal cell population of claim 6, wherein said cells are in contact with said liquid media for a period of time ranging from 2 hrs to 72 hours.

8. The adherent mesenchymal cell population of claim 5, wherein said administration is performed intramuscularly.
